(19) United States
(12) Patent Application Publication
(10) Pub. No.: US 2003/0105411 A1
(43) Pub. Date: Jun. 5, 2003

(54) Electrical impedance measuring method for differentiating tissue types

(76) Inventors: Rodney Harris Smallwood, Bradwell (GB); Brian Hilton Brown, Holmesfield (GB); Anthony David Blackett, Cawthorne (GB); Karen Julie Boston, Chesterfield (GB); John Anthony Tidy, Sheffield (GB)

Correspondence Address: Nixon & Vanderhye, 8th Floor, 1100 North Glebe Road, Arlington, VA, 22202-4714, US

(21) Appl. No.: 10/220,571

(22) PCT Filed: Mar. 2, 2001
(86) PCT No.: PCT/GB01/00907

(30) Foreign Application Priority Data
Mar. 3, 2000 (GB) 00O5247.2

(51) Int. Cl. A61B 5/05

(52) U.S. Cl. 600/547

(57) ABSTRACT

A method for differentiating tissue types, which is suitable as a method for obtaining data to enable a cancer screening process, comprises applying an alternating electric current to an area of tissue across a range of frequencies. The tissue impedance is measured at each frequency and the results fitted to a Cole equation. It has been found that the method is good at distinguishing between tissues having different size nuclei, or different ratios of nuclear to cytoplasm volume. This is related to the resistance (S) to electrical current flow through cytoplasm. Results may be improved by combining S with a value (R) for the resistance offered to electrical current through pathways between the cells. The method may be used in vivo or in vitro.

The invention relates to a method for differentiating animal or plant tissue types by measuring the electrical impedance of tissue. The invention also relates to a method of gathering data to enable screening for potential cancer or pre-cancer.

It is known to measure the electrical impedance if tissue to determine aspects of tissue structure. A technique is available known as “electrical impedance tomography” in which a number of impedance readings are taken at spaced apart locations on a region of the human body and an image derived from the data.

The inventor of the present invention has published a paper (IEEE/EMBS 20th Int. Conf. Hong Kong 2886-2889) describing a method of differentiating tissue types using impedance measurements over a range of frequencies. The main thrust of the paper was to determine whether “in vivo” impedance measurements on different tissues using a specially designed probe matched up to those predicted by electrical models consisting of networks of capacitors and resistors. The models predicted different electrical parameters for the different tissue types which should allow the tissue types to be differentiated “in vivo”.

A probe was developed comprising a rod of diameter 5.5 mm with a tetrapolar electrode arrangement, the electrodes being flush with the probe tip. It was desired to assess the probe's potential for differentiating different types of normal tissue and for differentiating normal tissue from cancerous or pre-cancerous tissue. To this end, the probe was used on a limited sample of subjects with areas of suspected cancerous or pre-cancerous cervical tissue and impedance values recorded for eight positions on the cervix for each subject.

Reasonably large samples were taken of normal tissue types (between 40 and 80 readings for each type) and good separation achieved in the readings for normal squamous epithelial and columnar tissues. A much smaller sample of readings from cancerous tissue was taken (12 readings) and a good degree of separation from normal squamous epithelial tissue obtained.

The impedance readings were fitted to a so called Cole equation which is known in itself and which takes the form:

$$Z = {R}_{∞} + \frac{( {R}_{0} - {R}_{∞} )}{1 + {( jF / {F}_{c} )}^{( 1 - α )}}$$

where:

Z=Impedance (Ohms)

R_{∞}=Resistance at infinite frequency (Ohms)

Rₒ=Resistance at d.c. (Ohms)

F=Frequency (Hertz)

F_{c}=The “critical frequency” (Hertz)

α=A so called “distribution constant” (dimensionless)

The impedance measurements were taken by applying a continuous a.c. current of 10 μA p-p for short periods of time over a frequency range of 9.6 kHz to 1.2 MHz. It was known that this frequency range would include a so-called “critical frequency”. Roughly speaking, this is the frequency at which the passage of current across cell membranes in the tissue, and thence through the conductive intracellular fluid (cytoplasm), becomes significant. Cell membranes are electrically insulating and therefore in electrical terms constitute capacitors.

From the fitted results it is possible to deduce a value R, the resistance of the conduction path between cells in the tissue, which is essentially non-capacitative. This will be termed the “extracellular resistance”. It is equivalent to Ro in the Cole equation. It is also possible to deduce Fc, a constant representing a critical frequency value related to the capacitance created by the cell membranes, and S which represents the electrical resistance inside the cells.

The results presented in the paper show how two different normal tissue types can be distinguished using Fc and R and how normal squamous tissue can be distinguished from cancerous tissue. In the discussion section at the end of the paper the separation of the two normal tissue types, squamous epithelial and columnar, is said to be good. It is also mentioned in this section that the best separation of these normal tissues was given by using the ratio of R to S, although these results are not presented. It should be noted that these normal tissue types have similar sized cell nuclei and similar ratios of nuclear to cytoplasmic volume—this is important to understanding the nature and significance of the invention with regard to this published paper which constitutes the closest prior art to the present invention.

Another type of probe has been developed for use in screening for pre-cancerous changes in cervical tissue. This is known as the “polarprobe”. This probe has three electrodes, and works by pulsing a short duration current (˜100 μS-200 mS) through the tissue and then monitoring the decay of charge, which gives an indication of capacitance. The polarprobe does not measure R or S. Clinical trials using this probe are currently underway but have not yet been reported fully.

The present invention has arisen from continued work with the tetrapolar probe discussed above, as opposed to the polarprobe. It has been found that unexpectedly good separation of tissue types can be achieved when one of the tissues contains larger nuclei than the other type, in proportion to the cell size. Another way of defining this difference is that the ratio of nuclear to cytoplasmic volume for the two tissue types are substantially different. It has been found that the value for S, the intracellular resistance, is significantly affected by the relative sizes of the nucleus and cell in a given tissue, and therefore that deriving a value for S provides an excellent method for differentiating tissues where the nuclear volume to cytoplasm volume ratio differs. The results may be further improved by combining the S values with the values for R, the extracellular resistance, which will generally be different for any different tissues.

A theoretical basis for these unexpectedly good results is proposed by the inventor, which is that up to a certain frequency the current will not “penetrate” the nucleus, since its membrane is capacitative. In fact, although the nuclear membrane itself probably has approximately the same specific capacitance as the cell membrane, the shape and dimensions of the nucleus mean that the critical frequency for the nuclear membrane is substantially higher than that for the cell membrane. There is therefore a range of frequencies, starting at about 50 kHz and extending beyond 1 MHz, where the value of the resistance S of the cytoplasm has a significant effect on the overall impedance of the tissue, and the resistance of the material within the nucleus has little or no effect on the overall tissue impedance because little current will penetrate the nucleus.

It may be that the results could be improved by increasing the top end of the frequency spectrum to values well above 1 MHz where there would be significant current flow through the nucleus. The results could then be analysed to give a third resistive value T representing the nuclear resistance. This result could be combined with S, and perhaps R as well. T would, in theory, decrease as the ratio of nuclear volume to cytoplasm volume increases whilst S would increase.

It is known to use frequencies in the range 500 MHz and above to obtain data related to molecular structures in human and animal tissue. Similarly, as said before, it is know to use frequencies appropriate for measuring R. What is not generally known, although it is disclosed in the paper referred to above, is the measurement of S by using frequencies which penetrate the cell membrane but not the nuclear membrane. The measurement of T, as mentioned above, has not been disclosed to the applicants' knowledge, and it is probable that there are other resistance values obtainable by increasing the frequency to points where other capacitance barriers are penetrated.

The method of the invention has been used on a sample of subjects with suspected cervical cancer or pre-cancer with excellent results. In cancerous and pre-cancerous tissues there is a marked increase in the size of the cell nuclei as well as changes in cell shape and size and the arrangement of the cells making up the tissue. However, there is no reason to believe that similar results would not be achievable with other tissue samples where the nuclear volume to cytoplasm volume ratio is very different between two tissue types in the sample. It should also be noted that increase in nuclear volume with respect to cytoplasm volume is observed in most if not all cancerous and pre-cancerous epithelial cells, whether they be columnar or squamous.

Reference is made to an article by V. Backman et al (Nature, Vol 406, Jul. 6, 2000) discussing an optical method for differentiating cancerous and pre-cancerous epithelial tissue from normal epithelial tissue. The article discusses the enlarged nuclei in the cancerous and pre-cancerous tissues, and experimental data is presented for oesophageal, colon, urinary bladder and oral cavity epithelia.

It is therefore to be expected that the impedance methodology presented here will be effective in all epithelial tissue, and at least in the four areas of epithelial tissue mentioned in the Backman reference in addition to cervical epithelium.

There is no reason to believe that this technique could not be used on a biopsy sample, i.e. in an “in vitro” situation as well as in an “in vivo” situation.

The theoretical basis for these results was tested by using a finite element model of an area of cervical squamous epithelium. The model was used to calculate the real component of the impedance over a frequency range of 100 Hz to 10 MHz, and this was done for a normal tissue model and for theoretical cases where the ratio of nuclear volume to cytoplasm volume increased. As expected, the curves on the impedance v. frequency plot were coincident up to a frequency of a few 10 s of kHz, at which point the plots for the normal tissue and the enlarged n:c (nuclear:cytoplasm) ratio tissue began to diverge.

The present invention in one aspect is a method of differentiating in a given area of tissue two or more tissue types whose cells have nuclei of different sizes, the method comprising the steps of:

(a) applying an alternating electric current to the area of tissue across a range of frequencies;

(b) measuring the tissue impedance at each frequency;

(c) deriving from the results an intracellular resistance value S representing electrical resistance offered by cytoplasm; and

(d) differentiating the tissue types based on the value S.

Its is found that the value for S provides an unexpectedly good separation of tissue types with different size nuclei.

Alternatively the above method could be defined as a method for differentiating tissue types having substantially different nuclear to cytoplasmic volume ratios.

The range of frequencies preferably includes one or more values above 20 kHz, preferably between 50 kHz and 1.5 MHz, more preferably 100 kHz and 1 MHz, still more preferably 300 kHz and 1 MHz, still more preferably 500 kHz and 1 MHz.

Preferably, the method further comprises:

(a) deriving from the fitted results an extracellular resistance value R representing electrical resistance offered by extracellular current pathways in the tissue; and

(b) differentiating the tissue types based on a combination of the values R and S.

R is known to be a good differentiator of tissue types in general, being a measure of the resistance offered by the extracellular current paths. Combining S and R gives excellent separation where different nuclear sizes occur in different tissues which also have different overall structure, i.e. different shapes and/or arrangements of cells.

The frequency range preferably also includes one or more discrete frequencies between 1 Hz and 50 kHz, preferably 1 kHz and 20 kHz. These frequencies provide a value for R. Thus, to obtain values for R and S, the lower end of the frequency range is preferably in these value ranges, whilst the upper end of the frequency range is preferably above 500 kHz, more preferably 700 kHz, still more preferably 1 MHz.

Also according to the invention there is provided a method of screening for the presence of potentially cancerous or pre-cancerous tissue comprising cells having enlarged cell nuclei, the method comprising:

(a) bringing into contact with a living human or animal subject a device for applying an alternating electric current, and applying a current to an area of tissue across a range of discrete frequencies;

(b) measuring the tissue impedance at each frequency;

(c) removing the said device from the subject;

(c) deriving from the results an intracellular resistance value S representing electrical resistance offered by cytoplasm; and

(d) deciding whether further investigation by biopsy or another method is required, based on the value S.

Good separation of pre-cancerous from normal tissue has been possible with this technique. The technique therefore lends itself to a method for screening for pre-cancer, where changes in nuclear size are observed. In the work which has been done to date, raw impedance data has been extracted from the device whilst in use and recorded. The device is then removed from the subject and the data processed by computer to derive a value for S. It is possible from the value for S to determine whether further more time consuming procedures are needed to establish the presence of pre-cancerous tissue. Final verification of the presence of tissue with the potential to develop into cancer is always provided by colposcopy and/or biopsy, of course. The usefulness of this procedure is as a screen to screen out cases which are “normal” from those requiring further assessment.

The method lends itself particularly to screening for cancerous or pre-cancerous epithelial tissue.

The range of frequencies preferably includes one or more values above 20 kHz, preferably between 50 kHz and 1.5 MHz, more preferably 100 kHz and 1 MHz, still more preferably 300 kHz and 1 MHz, still more preferably 500 kHz and 1 MHz.

The method advantageously further comprises:

(a) deriving from the results an extracellular resistance value R representing electrical resistance offered by current pathways between cells in the said area of tissue; and

(b) making said decision on the requirement for further investigation based on a combination of the values R and S.

Pre-cancerous tissue also has altered overall structure: the shapes of the cells and their arrangement changes. Accordingly, R is also a good indicator that pre-cancerous tissue may be present, and the combination of R and S gives even better results.

The frequency range preferably also includes one or more discrete frequencies between 1 Hz and 50 kHz, preferably 1 kHz and 20 kHz. These frequencies provide a value for R. Thus, to obtain values for R and S, the lower end of the frequency range is preferably in these value ranges, whilst the upper end of the frequency range is preferably above 500 kHz, more preferably 700 kHz, still more preferably 1 MHz.

In the work which has been done, it has been found that reasonably good values for R and S may be achieved by fitting the impedance data at different frequencies to a Cole equation of the form:

$$Z = {R}_{∞} + \frac{( {R}_{0} - {R}_{∞} )}{1 + {( jF / {F}_{c} )}^{( 1 - α )}}$$

where:

Z=Impedance (Ohms)

R_{∞}=Resistance at infinite frequency (Ohms)

Rₒ=Resistance at d.c. (Ohms)

F=Frequency (Hertz)

F_{c}=The crititcal frequency (Hertz)

α=A dimensionless constant.

The device is under development and it is anticipated that in time it will be possible to provide results on which a positive diagnosis may be made, and this may be performed in vivo or on a biopsy sample. In fact there is no reason to suppose that this would not be possible since similar techniques for deriving R have been employed on biopsy samples without undue difficulty.

According to the invention there is provided a method of analysing tissue biopsy samples, preferably epithelial tissue biopsy samples, for the presence of cancerous or pre-cancerous tissue comprising cells having enlarged cell nuclei, or having a nuclear to cytoplasmic volume ratio differing substantially from normal the method comprising:

(a) taking a tissue biopsy from a human or animal;

(b) applying an alternating electric current to the tissue across a range of discrete frequencies;

(c) measuring the tissue impedance at each frequency;

(d) deriving from the results an intracellular resistance value S representing electrical resistance offered by cytoplasm; and

(e) determining the probability of cancerous or pre-cancerous tissue being present based on the value S.

The range of frequencies preferably includes one or more values above 20 kHz, preferably between 50 kHz and 1.5 MHz, more preferably 100 kHz and 1 MHz, still more preferably 300 kHz and 1 MHz, still more preferably 500 kHz and 1 MHz.

The method advantageously further comprises:

(a) deriving from the results an extracellular resistance value R representing electrical resistance offered by current pathways between cells in the said area of tissue; and

(b) making said determination of the probability of cancerous or pre-cancerous tissue being present based on a combination of the values R and S.

The frequency range preferably also includes one or more discrete frequencies between 1 Hz and 50 kHz, preferably 1 kHz and 20 kHz. These frequencies provide a value for R. Thus, to obtain values for R and S, the lower end of the frequency range is preferably in these value ranges, whilst the upper end of the frequency range is preferably above 500 kHz, more preferably 700 kHz, still more preferably 1 MHz.

Further features and advantages of the invention will be apparent from the following description of the work that has been done to date, which refers to the accompanying drawings in which:—

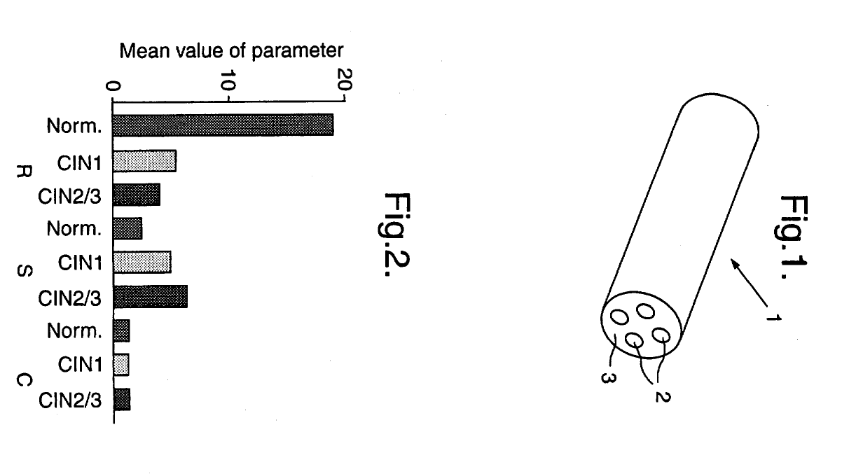

FIG. 1 is a perspective view of the end portion of a probe used in a method according to the invention;

FIG. 2 is a histogram showing the mean values for the Cole parameters R, S and C in an in vivo experiment;

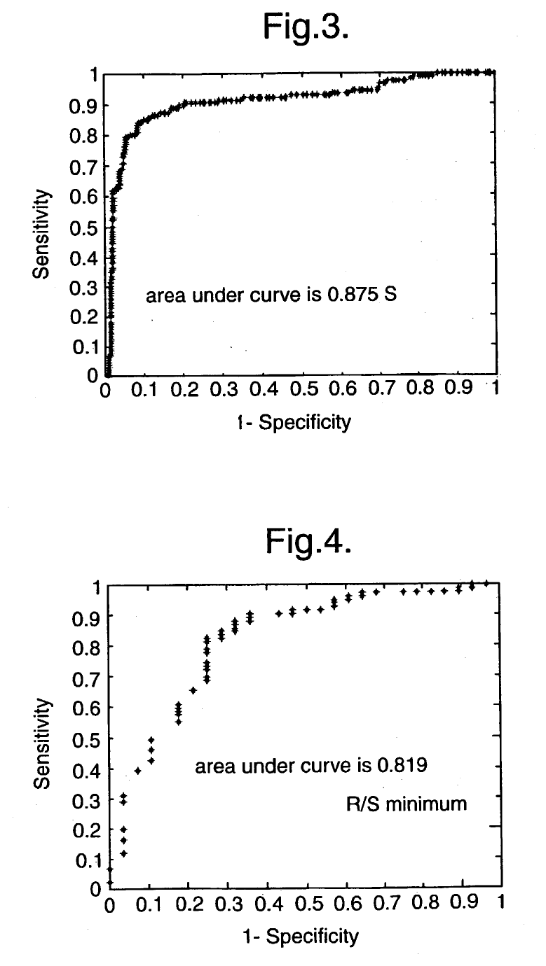

FIG. 3 is an ROC curve derived from data for S from the in vivo experiment;

FIG. 4 is an ROC curve showing a “per woman” comparison using data for R/S from the in vivo experiment;

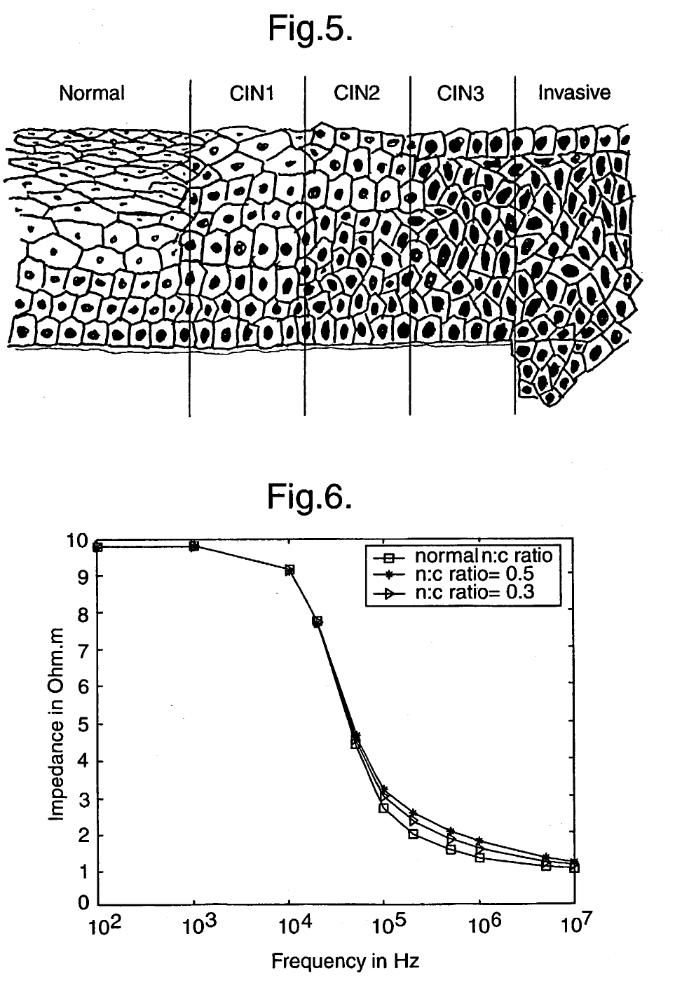

FIG. 5 is a diagram of epithelial tissue showing the progression from normal to invasive cancer; and

FIG. 6 is a plot of impedance (real component) v. frequency for a finite element model of cervical squamous epithelium, showing changes for different ratios of nuclear to cell sizes;

The Probe

Impedance measurements were made using a 5·5 mm-diameter pencil probe 1, with four 1 mm diameter gold electrodes 2 mounted flush with the end face 3 of the probe and spaced equally on a circle of radius 1·65 mm (FIG. 1). A current of 10 μA peak-to-peak was passed between an adjacent pair of electrodes and the real part of the resulting potential was measured between the two remaining electrodes. The ratio of the measured potential to the amplitude of the imposed current determines a transfer impedance. Measurements were made at eight frequencies by doubling the frequency in steps between 4·8 kHz and 614 kHz. Measurements were made serially at 67 frames per second and input to a computer. In nearly all cases two separate sets of data (each of 100 measurements recorded over 1·5 seconds) were recorded in succession in order to check reproducibility of the measurements. Only the first set of measurements are used for the results presented in Tables 1 & 2. Calibration was performed by placing the probe in saline of known electrical conductivity. A 4-electrode measurement of the transfer impedance spectrum is essentially independent of the contact impedance between electrode and tissue (which is of the order of 1 kΩcompared to the transfer impedance of roughly 100 Ω).

Subjects

The majority of subjects were women who had Pap smear results indicating moderate or severe dyskaryosis. However, three women with borderline changes and two with mild dyskaryosis were also studied. Impedance measurements were made before acetic acid was applied for the purposes of colposcopy. The probe was placed in eight positions on the cervix. These were as for the cardinal points of the compass with four positions close to the border with the endocervical canal and the remaining four well into the normal squamous epithelial surface of the cervix. Colposcopy examinations, including probe positioning, were recorded by video to allow for correlation between results obtained from colposcopic impression, histopathological examination of colposcopically-directed punch biopsies and the impedance measurements.

A clear colposcopy result and good impedance data were available for 756 measurements made on 124 women. The maximum possible number of measurements was 8×124 i.e. 992. In 221 cases the tissue, at the point where the probe had been placed, was not clearly identified either by biopsy or colposcopy. A further 15 measurements were rejected on technical grounds. In nearly all cases this was because the probe moved during data collection.

After comparing colposcopic and histology results there were found to be 370 measurements from normal squamous epithelium, 1 from an invasive cancer, 126 from CIN 2/3 (high grade) and 63 from CIN 1 (low grade). In addition 64 points were classified as mature metaplasia, 98 as immature metaplasia and 34 as columnar tissue. To qualify as ‘normal’ squamous epithelium had to lie outside the transformation zone, show no evidence of change with acetic acid and have a positive staining with Lugol's iodine.

Analysis

The 100 measurements forming the first data set recorded at each measurement position were averaged to give mean values of impedance at each of the 8 frequencies. These data, forming an impedance spectrum, were then fitted by a least square deviation method to a Cole equation of the form:

$$Z = {R}_{∞} + \frac{( {R}_{0} - {R}_{∞} )}{1 + {( jF / {F}_{c} )}^{( 1 - α )}}$$

to give estimates of R₀, R_{∞} and F_{c}. R₀ and R_{∞} are the impedances (real part) at very low and very high frequencies respectively, F_{c} is a frequency and α is a constant. α increases with the inhomogeneity of tissue but we assumed a value of zero as this was found to improve accuracy in the estimation of F_{c}. In this case an equivalent electrical circuit consisting of a resistor R placed in parallel with a resistor S and capacitor C in series will have an impedance Z, given by the above equation, where:

$${R}_{0} = R , {R}_{∞} = \frac{RS}{R + S} , {F}_{c} = \frac{1}{2 π C ( R + S )}$$

Parameters R, S and C can thus be determined from the fitted Cole equation. Because the probe was calibrated in saline of known conductivity, R and S are inversely proportional to conductivity and have the units of Ωm. They can be related to the extracellular and intracellular spaces respectively. C is related to the cell membrane capacitance and is given in units of μFm⁻¹.

The Cole equation is not the only method of analysing this sort of data to give values for R, S and F_{c} or C. There are other more sophisticated methods known in this art which may in fact give more accurate values for R, S and F_{c} or C.

Results

The derived Cole equation parameters R, S and C for the four tissue groups are given in Table 1. A range of statistical parameters are also given. 95% confidence levels on the means (95% CI) are given for guidance but these assume the distributions to be Gaussian. Non-parametric Mann Whitney two-tailed tests were also performed and showed that there are several significant separations of the groups. The values for R and S separate normal squamous epithelium tissue from the CIN 2/3 tissues (p<0·0001 in both cases). R and S also separate normal squamous epithelium from CIN 1 tissues (p<0·0001 in both cases). S separates CIN 1 from CIN 2/3 tissues (p=0·0009). The values for C do not show any significant changes. There was only one tissue sample corresponding to invasive cancer. The measurements in this case were 8·0, 5·1 and 0·28 for R, S and C respectively.

The two repeated blocks of 100 measurements were used to check the reproducibility of the measurements. The coefficients of variation (standard deviation/the mean) in the measurements were 0·108, 0·263 and 0·253 for R, S and C respectively.

In order to assess the statistical independence of the estimated values for R and S a Pearson correlation was performed using the pooled data for normal squamous epithelium, CIN1 and CIN 2/3 tissues. r² was 0·086 which can be interpreted as showing that only 8·6% of the variation in R can be attributed to variations in S and vice versa.

The changes in R and S as the epithelia progress from normal squamous through CIN 1 to CIN 2/3 are illustrated by the histograms given in FIG. 2 and can be summarised as follows:

R decreases by about a factor of 5.

S increases by about a factor of 2·5.

C does not change.

In order to help understand the possible utility of the technique as a screening test Receiver Operating Characteristic (ROC) curves have been derived for the normal squamous epithelium and CIN 2/3 tissue groups (FIG. 3). ROC curves show the sensitivities (1—the fraction of false negatives) and specificities (1—the fraction of false positives) obtained in using the two parameters R and S as discriminants between the normal squamous epithelium and CIN 2/3 tissue groups. If the measurements give no discrimination between the two groups then a single line at 45° is obtained. If there is a discrimination then the curve is displaced upwards and to the left. The area under the curves is given. An area of 0.5 corresponds to no discrimination between the groups and an area of 1.0 to perfect separation.

Analysis Per Woman

In addition to the analysis of the data on the basis of each measurement site the data were also grouped for each woman. This was carried out in order to make comparisons between the electrical impedance measurements and the results of both the referral smear test and the outcome of the colposcopy examination.

In order to provide a single indicator for each woman R/S was first calculated for each of the eight measurement sites. This was an attempt to take into account the fact that R decreases and S increases as we progress from normal squamous epithelium through CIN1 to CIN2/3 . Other combinations of R and S could be used and no attempt has been made to optimise separation of the tissues on this basis. The lowest value of R/S (R/S minimum) was then taken as the outcome for each woman on the basis that this should identify the greatest abnormality. However, it was observed that this method of identification included a number of tissue sites which were identified by colposcopy as columnar or immature metaplasia tissues. In order to reduce this confusion, sites where R was less than or equal to 2·36 Ωm (the 25% percentile for the CIN2/3 group) were excluded when taking the minimum value of R/S in each woman.

The colposcopy and biopsy results were used to place women into either a CIN group or a ‘normal’ group. All of the ‘normal’ group had at least two colposcopic examinations, six months apart, with repeat cervical cytology and biopsies. If any tissue of CIN1 or CIN2/3 was identified then the woman was placed in the CIN group. There were 88 in the CIN group and 28 in the ‘normal’ group. 8 were excluded from the total of 124 women. Women were excluded if we obtained fewer than six out of the possible eight measurements or the outcome of the colposcopy investigation was ambiguous.

The R/S minimum results are compared with the CIN, ‘normal’ classification using the ROC curve given in FIG. 4. The area under this curve is 0·819. Table 2 gives a range of statistical parameters derived from these data.

If we categorise the 116 women on the basis of the impedance results and use the 75% percentile point (0·81) for R/S minimum as the borderline then impedance produced the following performance measures: sensitivity 75% (66/88), specificity 71% (20/28), positive predictive value 89% (66/74) and negative predictive values 45% (20/44). In this study cervical cytology had a positive predictive value of 76% (88/116). No other measures could be calculated because all the women had positive smear results.

TABLE 1

| | Normal | | | | | | | | |
|---|---|---|---|---|---|---|---|---|---|
| | Squamous | | | | | | | | |
| Tissue | Epithelium | | | CIN 1 | | | CIN 2/3 | | |
| Parameter | R | S | C | R | S | C | R | S | C |
| Number of values | 370 | 370 | 370 | 63 | 63 | 63 | 126 | 126 | 126 |
| Minimum | 1.45 | 0.03 | 0.06 | 0.69 | 0.08 | 0.12 | 0.89 | 0.77 | 0.05 |
| 25% percentile | 12.8 | 1.15 | 0.37 | 2.69 | 2.49 | 0.33 | 2.36 | 4.39 | 0.36 |
| Median | 20.1 | 1.91 | 0.65 | 3.27 | 4.53 | 0.66 | 2.98 | 6.08 | 0.64 |
| 75% percentile | 26.8 | 2.78 | 1.20 | 5.52 | 6.31 | 1.46 | 4.22 | 7.63 | 1.09 |
| Maximum | 28.8 | 73.8 | 27.4 | 28.8 | 12.5 | 6.02 | 21.7 | 13.0 | 19.3 |
| Mean | 19.0 | 2.31 | 1.12 | 5.36 | 4.79 | 1.01 | 3.85 | 6.10 | 1.01 |
| Std. Deviation | 7.77 | 4.04 | 1.96 | 5.84 | 3.09 | 1.01 | 2.89 | 2.57 | 1.93 |
| Std. Error | 0.40 | 0.21 | 0.10 | 0.73 | 0.38 | 0.12 | 0.25 | 0.22 | 0.17 |
| Lower 95% CI | 18.1 | 1.90 | 0.92 | 3.88 | 4.02 | 0.76 | 3.34 | 5.64 | 0.67 |
| Upper 95% CI | 19.8 | 2.72 | 1.32 | 6.83 | 5.57 | 1.27 | 4.36 | 6.55 | 1.35 |

TABLE 2

R/S minimum

| | CIN | ‘normal’ |
|---|---|---|
| Number of values | 88 | 28 |
| Minimum | 0.2500 | 0.2900 |
| 25% percentile | 0.3500 | 0.8050 |
| Median | 0.4900 | 1.885 |
| 75% percentile | 0.8150 | 5.730 |
| Maximum | 8.200 | 12.10 |
| Mean | 0.8553 | 3.456 |
| Std. Deviation | 1.241 | 3.248 |
| Std. Error | 0.1323 | 0.6139 |
| Lower 95% CI | 0.5923 | 2.197 |
| Upper 95% CI | 1.118 | 4.716 |

The major changes in cervical tissue in the pre-cancerous stages are the breaking down of superficial cell layering and increases in the size of cell nuclei. This is illustrated in FIG. 5.

The Cole equation which has been fitted to the measured impedance spectra provides the parameters R, S and C. R is determined by the conduction pathways through the extracellular space and is hence sensitive to the packing of cells into layers. In normal squamous epithelium we would expect to see a high value for R as current has to track around the cell layers and hence take a long resistive path. In tissue graded as CIN 1 and CIN 2/3 the superficial cell layering of normal squamous epithelium is absent and hence R is greatly reduced. The observed changes in R fit well with this model; this outcome is consistent with what is already known.

S is determined by the conduction path through the intracellular space. The increase in S for CIN2/3 tissue appears to reflect the increased nuclear size in this tissue compared with normal. This has not previously been observed.

C is determined by the structure of the cell membrane. There is no evidence from the literature to enable a prediction to be made as to the changes expected in CIN 2/3 tissue.

The secondary objective of the work was to assess the potential of the technique as a method of screening for possible pre-cancerous changes in the female cervix. Our results show a very good separation of the measurements made on normal squamous epithelium and on CIN 1 and CIN 2/3 graded tissues. ROC curves (Figure) show that sensitivities and specificities of 0·9 can be obtained in detecting the changes associated with CIN 2/3.

Mitchell et al in the paper published in Obstetrics and Gynaecology, 93,3,462-470 (1999) reviewed several methods for the diagnosis of squamous intraepithelial lesions and derived ROC curves. They give areas under the ROC curves of 0·76 for Papanicolaou smear testing, 0·84 for diagnostic colposcopy and 0·71 to 0·75 for a fluorescence spectroscopy technique they have developed. In most cases Mitchell et al grouped CIN 1 and CIN 2/3 tissues together in deriving the ROC curves. The comparable figures for our data with CIN 1 and CIN 2/3 tissues grouped together are 0·934 and 0·834 for R and S respectively as the separating parameter. However, the comparison between these figures and those quoted by Michell et al may be misleading because our figures relate to measurements made at individual sites. A better comparison is to the ‘analysis per woman’ results which we present. The area under the curve for the ‘analysis per woman’ (FIG. 4) which used R/S is 0·819. This represents a considerable improvement over a Pap smear whilst being considerably more convenient and fast.

Finite Element Model

To provide further support for the conclusion that the parameter S is directly related to nuclear size, and/or to nuclear to cytoplasmic volume ratio, a finite element model of a 3 mm×3 mm×0.4 mm section of tissue was created. A simulation of the model having a current passed through it at a number of discrete frequencies was carried out, and the real component of the impedance measured at each frequency. This was repeated for changed ratios of nuclear:cytoplasmic volumes (n:c).

In the “normal” tissue, the ratio of n:c was 0.003 for the superficial cells, 0.02 for the intermediate cells, 0.04 for the parabasal cells and 0.27 for the basal cells. The ratios were then adjusted to 0.3 and then 0.5: these values equate approximately to the ratios in CIN 2/3 tissue. The results are shown in FIG. 6.

The results clearly show the curves coincident up to a value somewhere between 10 and 100 kHz at which point the curves separate, with the total real (ie resistive) impedance being higher for the higher ratios of n:c. This may be accounted for by the decreased conduction path through the cytoplasm where n:c is higher, giving a lower value for S and thus for total resistive impedance. The curves start to separate at the critical frequency at which the cell membrane is penetrated by the current.

These results were fitted to a Cole equation and values for R and S derived which equated well to the results obtained with real tissue.

In summary, the inventors have shown that some characteristics of the electrical impedance spectrum of tissue can be explained by changes in cell arrangements (layering) and in the size of the cell nuclei. This opens the way to deriving tissue structure from electrical impedance spectral measurements. For example, measurements might be made from the gastro-oesophageal junction and the bladder, where screening for pre-cancerous changes is of importance. We have shown that this methodology can be used to give good separation of cervical tissues. The sensitivities and specificities obtained are at least comparable with existing screening methods.

1. A method of differentiating in a given area of tissue two or more tissue types whose cells have nuclei of different sizes, the method comprising the steps of:
(a) applying an alternating electric current to the area of tissue across a range of frequencies;
(b) measuring the tissue impedance at each frequency;
(c) deriving from the results an intracellular resistance value S representing electrical resistance offered by cytoplasm; and
(d) based on the value S, differentiating the tissue types.

2. A method of screening for the presence of potentially cancerous or pre-cancerous tissue comprising cells having enlarged cell nuclei, the method comprising:
(a) applying an alternating electric current to an area of tissue across a range of frequencies;
(b) measuring the tissue impedance at each frequency;
(c) deriving from the results an intracellular resistance value S representing electrical resistance offered by cytoplasm; and
(d) based on the value S, making a determination as to whether further investigation by biopsy or another method is required.

3. A method of analysing tissue biopsy samples for the presence of cancerous or pre-cancerous tissue comprising cells having enlarged cell nuclei, the method comprising:
(a) taking a tissue biopsy from a human or animal;
(b) applying an alternating electric current to the tissue across a range of frequencies;
(c) measuring the tissue impedance at each frequency;
(d) deriving from the results an intracellular resistance value S representing electrical resistance offered by cytoplasm; and
(e) based on the value S, making a determination of the probability of cancerous or pre-cancerous tissue being present.

4. A method as claimed in any of claims 1 to 3 wherein the range of frequencies includes a frequency in the range 50 kHz to 1.5 MHz.

5. A method as claimed in any of claims 1 to 4 further comprising:
(a) deriving from the results an extracellular resistance value R representing electrical resistance offered by current pathways between cells in the said area of tissue; and
(b) making said determination or differentiation step based on a combination of the values R and S.

6. A method as claimed in claim 5 wherein the said combination of R and S is R/S or S/R.

7. A method as claimed in any preceding claim wherein the value S and/or the value R is derived by fitting the results to a Cole equation of the form:
$$Z = {R}_{∞} + \frac{( {R}_{0} - {R}_{∞} )}{1 + {( jF / {F}_{c} )}^{( 1 - α )}}$$
where:
Z=Impedance (Ohms)
R∞=Resistance at infinite frequency (Ohms)
Ro=Resistance at d.c. (Ohms)
F=Frequency (Hertz)
Fc=The crititcal frequency (Hertz)
α=A dimensionless constant.
